(19) United States
(12) Patent Application Publication
(10) Pub. No.: US 2023/0051427 A1
(43) Pub. Date: Feb. 16, 2023

(54) GALVANOSTATIC METHOD OF MICROBE REMOVAL FROM METAL ORTHOPEDIC DEVICES

(71) Applicant: Garwood Medical Devices, LLC, Buffalo, NY (US)

(72) Inventors: Jackson G. Hobble, Buffalo, NY (US); Brian R. Peterson, East Aurora, NY (US)

(21) Appl. No.: 17/793,580

(22) PCT Filed: Jan. 13, 2021
(86) PCT No.: PCT/US2021/013178

§ 371 Date: Jul. 18, 2022

Related U.S. Application Data
(60) Provisional application No. 62/962,524, filed on Jan. 17, 2020.

(51) Int. Cl. A61N 1/20; A61F 2/38; A61N 1/05

(52) U.S. Cl. CPC A61N 1/20; A61F 2/38; A61N 1/05; A61F 2002/30719

(57) ABSTRACT

A system and related method for eliminating microbes from a metal orthopedic appliance includes a counter electrode and working electrode used for applying treatment, and a reference electrode used for monitoring safety parameters. The working electrode is the implanted appliance. Electrical current is applied between the counter electrode and the working electrode to create electrochemical current through the system and create electrochemical reactions on the surface of the working and counter electrode. The chemical species created at the working electrode provide a mechanism to disrupt and kill microbes on that surface, including bacterial biofilms commonly found on infected orthopedic implants. Circuitry connected to the electrodes keeps the applied current constant and allows the voltage between the working and counter electrode to vary. The reference electrode monitors the voltage at the working electrode in order to provide feedback to a processor as part of a feedback mechanism. The processor is programmed with software logic, preventing the voltage from drifting to ranges that correlate with metal immunity or corrosion regions by limiting or altering the applied current.

### CROSS REFERENCE TO RELATED APPLICATION

This application is a national stage application under 35 U.S.C. § 371 of International Application No. PCT/US2021/013178, filed Jan. 13, 2021, which claims priority under applicable portions of 35 U.S.C. § 119 to U.S. Patent Application Ser. No. 62/962,564, filed Jan. 17, 2020, the entire contents of each application being herein incorporated by reference herein.

### TECHNICAL FIELD

This application is directed to a system and related galvanostatic method that eradicates microbes from the surfaces of metal orthopedic devices/appliances.

### BACKGROUND

Orthopedic devices, such as metal implants, are used in patients with many different injuries or medical problems. In particular, metal implants may be used for any individual that needs to replace joints. For example, a metal implant may be used to replace a patient's hips or knees. One potential problem with metal implants is that they tend to allow for the growth of bacteria on the surface. This may increase the patient's risk for an infection, which may result in removal of the implant or a life-threatening situation if the infection cannot be treated. To decrease the risk of infection, electrodes can provide electrical stimulation to disrupt the growth of bacteria.

It has been shown in scientific literature that application of cathodic current to metal samples create chemical reactions at that surface that can disrupt and kill bacterial biofilms that exist on the metal. For electrochemical processes to occur, there must be an anode and a cathode within an electrolyte solution. The anode is a metallic surface where oxidative reactions occur, and the cathode is another metallic surface where reduction reactions occur. A reduction reaction is essentially when the material of interest gains electrons and thereby decreases the oxidation state of the molecules. The electrolyte that the anode and cathode each reside in provides the electrical connection by facilitating the flow of electrons shuttled by ion carriers such as sodium or potassium ions. Electrons are driven from the anode to the cathode through the electrical path via an external power source such as a galvanostat. A galvanostat is an instrument used to drive constant current from a counter electrode to a working electrode by varying voltages between them. In the case of cathodic current stimulation, the anode represents the counter electrode and the cathode represents the working electrode.

The two-electrode system described has been used in industry for many decades to produce different chemical byproducts from the electrolyte media. In many two electrode, direct current applications there are common problems with controlling or knowing what thermodynamic state the surface of the working electrode exists as. Depending on the voltage and pH of the system, the metal may be in a corrosive, passive, or immune state. Not knowing which state the surface exists in may cause undesired metal substituents to be released, which can cause adverse effects on the patient's health if a galvanostatic operation is applied in the body of a patient with a metal implant. This can occur in both voltage and current controlled two-electrode systems.

### BRIEF DESCRIPTION

The invention is directed to curing the above noted problems in order to realize a galvanostatic technique that can safely remove microbes from the surface of an orthopedic appliance such as a replacement knee, shoulder or hip. By implementing a third electrode with a stabilized voltage as part of a feedback mechanism, the inventive system and method is able to sense what voltage the working electrode exists at under a galvanostatic system, and based on the sensed voltage levels, voltage limiters can be applied in order to prevent drift into thermodynamically unfavorable potentials. The inventive method and system is therefore comprised of three (3) electrodes including a counter electrode and working electrode used for applying treatment, as well as a reference electrode used for monitoring safety parameters. According to a preferred version of the inventive method and system, The working electrode is the surgically embedded implant. A flow of electrical current is applied between the counter electrode and the working electrode in order to create electrochemical current through the system. The applied electrical current is a direct current so to create electrochemical reactions on the surface of the working and counter electrode. The chemical species which are created at the working electrode provide a mechanism to disrupt and kill microbes on that surface including bacterial biofilms commonly found on infected orthopedic implants. The circuitry connected to the electrodes keeps the applied current constant and allows the voltage between the working and counter electrode to vary. The reference electrode is configured to monitor the voltage at the working electrode in order to provide feedback to a processor, forming a feedback mechanism. The processor is programmed with software logic in order to prevent the applied voltage from drifting to ranges that correlate with metal immunity or corrosion regions by limiting or altering the current and therefore keep the measured voltage within a predetermined range.

By implementing a third electrode with a stabilized voltage, the inventive system is able to sense what voltage the working electrode exists at under a galvanostatic system, and the processor can then apply voltage limiters to prevent drift into thermodynamically unfavorable potentials.

The inventive system and method is unique because it combines a simplistic electronic application of galvanostatic stimulation with a smart feedback mechanism in order to provide a controlled and effective means to eliminate microbes, including biofilms, from orthopedic hardware.

The invention provides greater patient safety following surgically implanted hardware and reduces the need to remove implants due to infection and minimizes prevention of life-threatening incidents.

These and other features and advantages will be readily apparent from the following Detailed Description which should be read in conjunction with the accompanying drawings.

### BRIEF DESCRIPTION OF DRAWINGS

FIG. 1 is a schematic view of a system in accordance with aspects of the present invention.

FIG. 2 is a view of the system as applied to a patient in accordance with an exemplary embodiment; and

FIG. 3 is a flow chart detailing an exemplary method in accordance with aspects of the invention.

### DETAILED DESCRIPTION

The following describes a novel system and method that removes microbes from a surgically implantable orthopedic device, such as a knee or hip replacement in accordance with an exemplary embodiment. It will be understood, however that other embodiments or versions will be apparent based on the inventive aspects described.

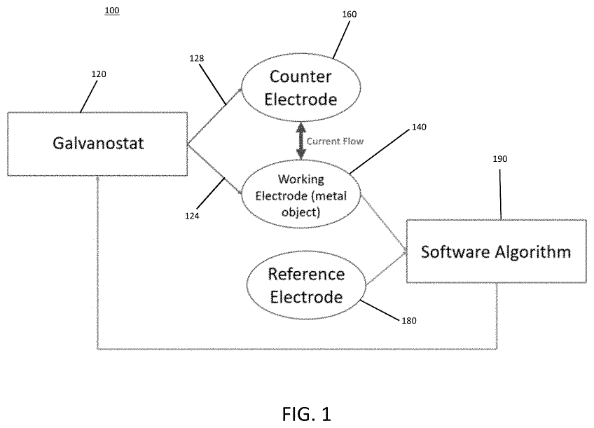

With reference to FIG. 1, there is shown a system shown schematically in accordance with an exemplary embodiment. The system 100 includes a galvanostatic device 120, which is coupled by electrical leads 124, 128 to a working electrode 140 and a counter electrode 160, respectively. As described in greater detail below, the number of working electrodes and counter electrodes can be suitably varied. For purposes of this invention, the galvanostatic device 120 can be any device, such as an amperostat that is configured to provide a flow of constant direct current through the attached working and counter electrodes 140, 160 and further configured so as to allow the voltage in the system 100 to vary. The working electrode 140 according to this embodiment is a surgically implanted orthopedic appliance, such as a knee or hip replacement, which is defined by a metal surface, such as titanium, zirconium, cobalt chrome, stainless steel or other metallic material and/or alloys. The counter electrode 160 can theoretically be any electrically conductive material, but preferably a material that remains chemically inert when acting as an anode (carbon or platinum). The working electrode 140 is further coupled, according to this embodiment, to a reference electrode 180, the latter being coupled to the galvanostatic device 120. The reference electrode 180 is configured to monitor the voltage of the working electrode 140 and provide an input signal representative of the monitored voltage to the galvanostatic device 120. The counter electrode 160 and the reference electrode 180 can both be either implanted or external to the patient, but are preferably external in the form of electrode pads that adhere to the skin. According to this embodiment, the reference electrode 180 is made from Ag/AgCl, although other materials can be used.

The galvanostatic device 120 according to this embodiment is configured with a processor that is programmed with logic (shown as 190) that compares the sensed voltage of the working electrode received from the coupled reference electrode 180 with a stored and predetermined range of voltages or a voltage maximum. It should be noted that the processor can be a separate device or can be integrated directly into the galvanostatic device 120, which defines a feedback mechanism for the herein described system. In use and if the monitored voltage exceeds the voltage maximum as detected by the reference electrode 180, then the galvanostatic device 120 is programmed to automatically vary the constant current to the coupled electrodes 140, 160 in order to limit the voltage of the system 100 and address thermodynamic safety concerns relating to the implanted orthopedic appliance.

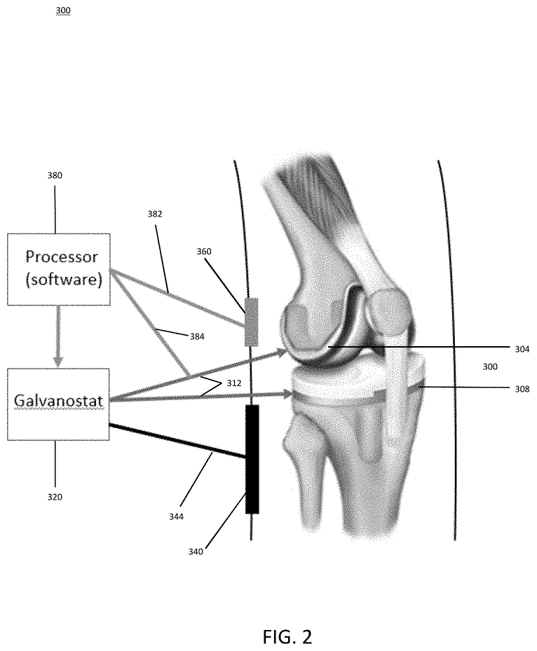

With reference to FIG. 2, a typical system in accordance with the present invention is shown a use condition and in conjunction with a specific implanted knee implant (replacement) of a patient. According to this embodiment, the implant 300 is a full knee replacement that includes respective femoral and tibial components 304, 308. As previously noted, the depicted system is an example as the inventive system is equally applicable to any metal orthopedic implant. As shown, a series of needles 312 extend from a galvanostatic device 320. Only a pair of needles are illustrated in this specific example, but it will be understood that the number of needles for treatment can be suitably varied. Each needle 312 represents a working electrode that connects to a different section of the metal implant 300, as shown, for application of current from the galvanostatic device 320. The galvanostatic device 320 as previously defined is a device that is configured to provide a flow of constant direct current through the attached working electrode, as well as an attached counter electrode 340 and is further configured so as to allow the voltage in the system to vary. The counter electrode 340, also shown schematically, is adhered by means of a pad to the skin of the patient along with the reference electrode 360, which is similarly adhered to the skin according to this version. Each of the needles 312, as well as the counter electrode 340, via lead 344, are coupled to the galvanostatic device 320, the latter being further coupled to a processor 380, shown herein as a separate device, which includes comparative logic. As previously noted, the processor 380 can alternatively be integrated as part of the galvanostatic device 320. The processor 380 is further coupled to the reference electrode 360 via lead 382 and includes an additional lead, shown schematically as 384 in this view, extending to the working electrode/needles 312. This additional lead 384 is provided for monitoring the voltage of the working electrode and providing an input signal to the processor 380, as previously described, in order to compare the voltage measurement of the working electrode(s) to a stored value or range of voltages.

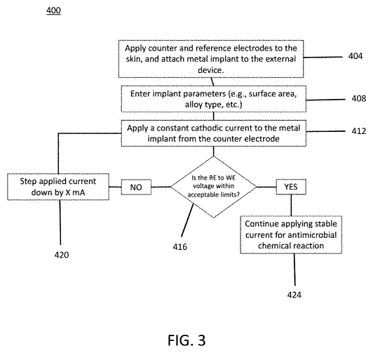

An exemplary method 400 in accordance with the invention is detailed with reference to FIG. 3. According to a first step 404, the reference and working electrodes are applied to the skin and the needles are attached to the metal implant and connected to the external galvanostatic device. Preferably and according to step 408, implant parameters including surface area, alloy type and the like are entered into the processor. A constant cathodic current flow can then be applied by the galvanostatic device between the working electrode and the counter electrode, according to step 412. The reference electrode monitors the voltage of the working electrode and inputs a signal to the processor. According to step 416, the processor determines whether the voltage to the working electrode is within stored limits, the latter being based on the implant parameters. If the comparison indicates that the voltage is within reasonable limits (a defined maximum voltage or range of voltages), then according to step 424, the stable current applied by the galvanostatic device is maintained in order to act upon the metal implant surface to eradicate microbes. If the comparison indicates that the applied voltage to the working electrode is greater than the stored values/ranges, then the current is reduced, per step 420.

Experimentation with clinical strength biofilms has shown that optimal current density upon a metal implant to remove at least three logs of bacteria over a period of time is 1-3 mA/cm²; however, the present system can be effective from 0.1 mA/cm² to infinite current density. Current density can become dangerous to the patient if dosed too aggressively. Duration of treatment in combination with current density can be optimized to provide the most effective kill of bacteria without harming the patient's own biological tissue. For example, a current density of 100 mA/cm² may cause high amounts of bone necrosis if applied for only a minute. Although current density is a simplistic way to baseline treatment parameters, total current is what simple galvanostats supply. As implant sizes vary due to the type of implant and size of the patient, a surface area calculation should be performed, as discussed above, in order to apply the optimal current density. For example, if invention needs to apply 2 mA/cm², to an implant that is 100 cm², the user must apply 200 mA through the invention for the desired treatment.

Typically, most metallic implants within the body are made from alloys that have the ability to passivate and create biocompatible oxide films at their surface under internal body environments and pH. Some examples of these metals include titanium, cobalt chrome, and stainless steel, among others. These biocompatible oxide films provide a kinetic barrier to prevent the metal from corroding into the external environment and thus provide the body, and in some cases biofilms, an inert surface to attach to. It is known that the thermodynamic equilibrium states of all metals can be modulated by changing their potential compared to a stable reference electrode, and the surrounding pH. Depending on the applied potential and the surrounding pH, metals may exist in passive, corrosive, or immune states. Passive states are largely considered safe. The previously mentioned oxide film that exist on titanium, cobalt chrome, and stainless steel is simply the metal thermodynamically existing in a “passive” state. As external potentials are applied in anodic or cathodic directions and electrolyte constituents change pH through chemical reactions, the passive layers may either grow or thin, respectively referred to as anodization or reductive dissolution. Depending on the metal, certain combinations of potential and pH can cause metals to enter thermodynamic states of corrosion or immunity. Corrosion states release metal ions into the surrounding environment whereas immunity states demonstrate non-corroding bare metal (with no oxide layer). Metal ions are known to cause unwanted side effects inside the body such as tissue necrosis or formation of pseudo tumors. The effect of immune metals on the body are not widely known, but are thought to cause biocompatibility and allergic reactions in surrounding tissue.

As discussed, concern has arisen regarding known two electrode systems and their lack of ability to control the thermodynamic state of the metal of interest. Without any feedback mechanism of what voltage the working electrode is at in comparison to a stable reference electrode, its potential may drift in anodic or cathodic directions, thus potentially entering corrosion or immune thermodynamic states. As discussed, the present system and related method adds a third (reference) electrode to its galvanostatic system that provides a feedback mechanism to a processor that is programmed with suitable logic that will alter the current of the galvanostatic device in order to prevent voltage drifts into corrosive or immune regions. Corrosive, passive, and immune regions naturally vary among all types of metal, therefore prior knowledge of the implants' metal composition is required, see step 408, FIG. 3, for the device to know what electric potentials can be considered safe for treatment. For example, titanium becomes immune when applied potentials exceed −1.8V vs. Standard Hydrogen Electrodes at an internal body pH of roughly 7. The reference electrode should be made from a material with a very stable voltage potential. In the preferred embodiment, the reference electrode is made from Ag/AgCl. While the stimulation is active and current is being passed from the counter electrode to the working electrode, the reference electrode is configured to monitor the working electrode's potential in reference to itself. This measurement can be fed back to the processor, either maintained separately or within the galvanostat external to the patient that can determine if the voltage is within or outside of the preferred voltage range for that type of metal. If the voltage is too cathodic, the processor is programmed to adjust the applied current to be an interval less anodic in order to bring the potential of the working electrode back with the acceptable range. This method of treatment puts priority on driving the correct current and makes adjustments if safety factors become an issue. This technique is superior as compared to a voltage controlled three-electrode system that pinpoints a specific voltage that may be within the acceptable range potential, but lacks consistency in its drifting current (the mechanism of treatment).

### PARTS LIST FOR FIGS. 1-3

100 system
120 galvanostatic device
124 lead, electrical
128 lead, electrical
140 working electrode
160 counter electrode
180 reference electrode
190 software algorithm (logic)
300 implant
304 femoral component, implant
308 tibial component, implant
312 needles
320 galvanostatic device
340 counter electrode
344 lead
360 reference electrode
380 processor
382 lead
384 lead
400 method
404 step
408 step
412 step
416 step
420 step
424 step

Other variations and modifications to the inventive system and method will be readily apparent to a person of sufficient skill.

1-14. (canceled)

15. A system configured for treating a metal orthopedic device, the system comprising:
a counter electrode and a working electrode, in which the metal orthopedic device acts as the working electrode;
a galvanostatic device coupled to the counter electrode and the working electrode and configured to apply a constant current flow between the counter electrode and the working electrode that further creates an electrochemical reaction on the surface of the metal orthopedic device capable of killing microbes formed on said surface; and
a feedback mechanism comprising a reference electrode and processor adapted to monitor a voltage at the surface of the metal orthopedic device, said processor being programmed to automatically adjust the constant current flow applied by the galvanostatic device based solely on the monitored voltage if the monitored voltage is outside a predetermined voltage range stored by the processor.

16. The system according to claim 15, wherein the processor is incorporated in the galvanostatic device.

17. The system according to claim 15, wherein the metal orthopedic device is a surgical implant.

18. The system according to claim 15, wherein the reference electrode and the counter electrode are disposed in relation to the metal orthopedic device.

19. The system according to claim 15, wherein the reference electrode is one of implanted or external to a patient having the metal orthopedic device.

20. The system according to claim 15, wherein the constant current flow is cathodic.

21. The system according to claim 15, wherein the reference electrode is made from silver/silver chloride.

22. The system according to claim 15, wherein the counter electrode is made from a chemically inert conductive material.

23. A method for treating a metal orthopedic device, the method comprising:
utilizing the metal orthopedic device as a first electrode;
providing a second electrode in relation to the first electrode;
electrically coupling the first and second electrodes to a galvanostatic device;
using the galvanostatic device, applying a constant current flow between the first and second electrodes in order to produce an electrochemical reaction capable of eradicating microbes from a surface of the metal orthopedic device; and
using a feedback mechanism having a processor:
monitoring the voltage of the metal orthopedic device using a reference electrode separate from the first and second electrode, wherein the reference electrode is coupled to the first electrode;
comparing the monitored voltage with a predetermined voltage range; and
using the processor, automatically adjusting the constant current flow between the first and second electrodes if the monitored voltage is outside the predetermined voltage range.

24. The method according to claim 23, including the step of disposing the processor in the galvanostatic device.

25. The method according to claim 23, wherein the metal orthopedic device is a surgical implant.

26. The method according to claim 23, wherein the reference electrode is one of implanted or external to a patient.

27. The method according to claim 23, wherein the constant current flow is cathodic.

28. The method according to claim 23, including the step of making the reference electrode from silver/silver chloride.

29. The method according to claim 23, including the step of making the counter electrode from a chemically inert conductive material.
